(19) United States
(12) Patent Application Publication
(10) Pub. No.: US 2004/0116779 A1
(43) Pub. Date: Jun. 17, 2004

(54) Method and tool for determining a physical or emotional state of a person

(76) Inventor: Denise Jean Bien, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/319,104

(22) Filed: Dec. 13, 2002

(51) Int. Cl. A61B 5/00

(52) U.S. Cl. 600/300

(57) ABSTRACT

A diagnostic tool for determining a state of person, the tool comprising at least one identified emotional or physical symptom and a series of visual indicia corresponding to each identified emotional or physical symptom. The visual indicia are preferably photographs, where the photographs are arranged in order of increasing communicated intensity. The user chooses one of the visual indicia as being representative of the user's felt intensity of the identified emotional or physical symptom. A numerical value can be associated with individual indicia, the numerical values can be used to evaluate the emotional or physical state of a person.

### FIELD OF THE INVENTION

This invention relates to methods and diagnostic tools for evaluating and determining the state of a person, for example the emotional or physical state.

### BACKGROUND

It is well known within the medical and mental health fields that a major obstacle to communication between people is a lack of emotional self-awareness and of an adequate vocabulary on the part of such individuals to permit the efficient or effective exchange of feelings or needs. This difficulty is particularly a problem, particularly if the health issues involve relatively private or difficult to discuss issues. One area in which it is difficult for health professionals to efficiently diagnose emotional or physical feelings relates to women's menstruation cycles and the emotional and/or physical feelings that accompany these cycles. For example, premenstrual syndrome (PMS) affects a large number of women, and effective treatment involves accurately determining a woman's emotional and/or physical feelings during this time. A particular difficulty faced by persons trying to determine intensity levels of emotional or physical feelings relating to PMS is separating out cyclical symptoms from ongoing symptoms.

It is also useful for doctors, clinicians, researchers, and other health professionals to have a tool by which individuals can record their subjective feelings related to various health issues. For example, with respect to women's health issues relating to PMS, menopause, or perimenopause, health professionals and researchers depend on clinical tests and consumer tests to design and tailor products to meet specific needs to control or reduce associated symptoms. These tests often rely on consumers completing a daily diary in which they record their subjective intensity levels relating to certain identified symptoms, both emotional and physical. Even when provided with certain identified symptoms, individuals often have difficulty adequately articulating the magnitude or intensity level of these feelings.

Several attempts at aiding individuals with identification of emotions and needs have been made, but so far there is no satisfactory solution to the larger problem of identifying and communicating both the severity of emotions felt and the resultant treatment with respect to adult women and their health needs.

Several U.S. patents teach the use of color-coded cards, blocks, shapes, flags or other devices to indicate the mood of a user. These patents include U.S. Pat. No. 5,580,254 to Ramsey (color-coded cards corresponding to a mood of a user); U.S. Pat. No. 5,040,988 to Brown (variety of flags and pegs for identifying various user moods); U.S. Pat. No. 4,173,833 to Rosenblatt (spheres of various colors); U.S. Pat. No. 5,471,137 to Aduvala (cards and blocks adapted for helping children name emotions); and U.S. patent application Ser. No. 2002/0,009,696 A1 (two sets of blocks bearing informative indicia identifying various emotions or feelings). In general, each of these tools and methods lacks the ability to not only identify many emotions, they lack the ability to facilitate the communication of the intensity of the emotions. For example, U.S. Pat. No. 5,741,137 uses cards labeled by certain attributes, with an emotional intensity indicator on the cards. The result is that, instead of the user assigning a level of personally-felt intensity, the cards dictate a given intensity that is simply assigned to each attribute. Likewise, U.S. application Ser. No. 2002/0,009,696 is represented as being helpful in improving an individual's ability to identify and express emotions, but it does not recognize the need to indicate levels of intensity of any given emotion. Another drawback to the methods disclosed above is that they primarily give a one-time evaluation, rather than a long term analysis that can identify long term changes and trends.

Current diagnostic tools used to evaluate women to determine and treat severity levels of physical and emotional feelings associated with PMS, for example, include “pre-screening” questionnaires which attempt to identify women with certain symptom severity levels to be studied with further treatment. “Baseline” diary studies can follow pre-screening questionnaires, in which women record their subjective feelings of intensity for identified emotional or physical symptoms in prospective daily diaries. The baseline diary studies can continue for 2 to 3 menstrual cycles. These diaries separate cyclical or periodic symptoms from chronic or ongoing symptoms that are present. If treatment follows, further diary studies can then be continued during treatment, with a comparison of the results with the baseline study. In this manner treatment can be regulated as needed.

Diaries, such as prospective daily diaries, can be used as a professional diagnostic tool, or as a self-diagnostic tool, and often require that the user fill in an oval, e.g., a “bubble”, corresponding to a quantifying scale. The scale may include a verbal range from “none” to “extreme” with the verbal scale being converted into a numeric scale for analysis. For example, on a scale having five intensity levels for a given symptom, the intensity levels can correlate to a numerical scale from 0-4. One method of diagnosis can comprise computing an average intensity level for all the symptoms, or an average for one or more of a group of related symptoms. By comparing the average on a daily basis, the health professional or products researcher can track changes during a menstrual cycle, for example. If the changes indicate a change in emotional or physical greater than a predetermined amount, say 30% for example, treatment may be indicated.

Current pre-screening and diagnostic tools, such as diaries, rely only on a numeric scale and/or a verbal scale to record and analyze intensity levels for symptoms. This can result in inaccurate information being relied upon and can result in less than ideal treatment. For example, often it is found with existing tools that the baseline study diary results do not correlate with the pre-screening questionnaire results. This is believed to be due to the fact that words or numbers alone are not sufficient to reflect severity levels of very subjective emotional or physical feelings. Another issue with the above “bubble” daily diary method is compliance. It is known that individuals tend to skip days or weeks of diary entries altogether or fill a complete week of diaries in one day.

Therefore, there remains a need for a diagnostic tool for determining the emotional or physical state of a person.

Likewise, there is need for a method of helping a person identify an emotional or physical symptom and communicate a level of felt intensity for that emotional or physical symptom.

Further, there is a need for a diagnostic method and tool that can correlate to daily diary data before and after treatment and thus provide a less complicated method of monitoring symptoms over time.

Finally, there is a need for a diagnostic method and tool that can correlate with Quality of Life questionnaires and pre-screening questionnaires thereby allowing the possibility of replacing currently used questionnaires with a simpler, less complex symptom severity assessment and monitoring method than currently used systems.

### SUMMARY OF THE INVENTION

A diagnostic tool for determining a state of a person is disclosed, the tool comprising at least one identified emotional or physical symptom and a series of visual indicia corresponding to each identified emotional or physical symptom. The visual indicia are preferably photographs, where the photographs are arranged in order of increasing communicated intensity. The user chooses one of the visual indicia as being representative of the user's felt intensity of the identified emotional or physical symptom. A numerical value can be associated with individual indicia, the numerical values can be used to evaluate the emotional or physical state of a person.

### BRIEF DESCRIPTION OF THE DRAWINGS

FIG. 1 shows a typical picture scale useful in a diagnostic tool of the present invention.

### DETAILED DESCRIPTION OF THE INVENTION

The diagnostic tool of the present invention can be used to determine the state of a person. By “state of a person” is meant that a person's physical or emotional state, which can be determined based on an evaluation of the intensity or severity level of one or more symptoms associated with the physical or emotional state of the person. The diagnostic tool of the present invention comprises at least one identified symptom, and a series of visual indicia associated with each identified symptom. Each individual indicium can correspond to a numerical value, and multiple identified symptoms can be analyzed to get a numerical rating for a given person and a given state.

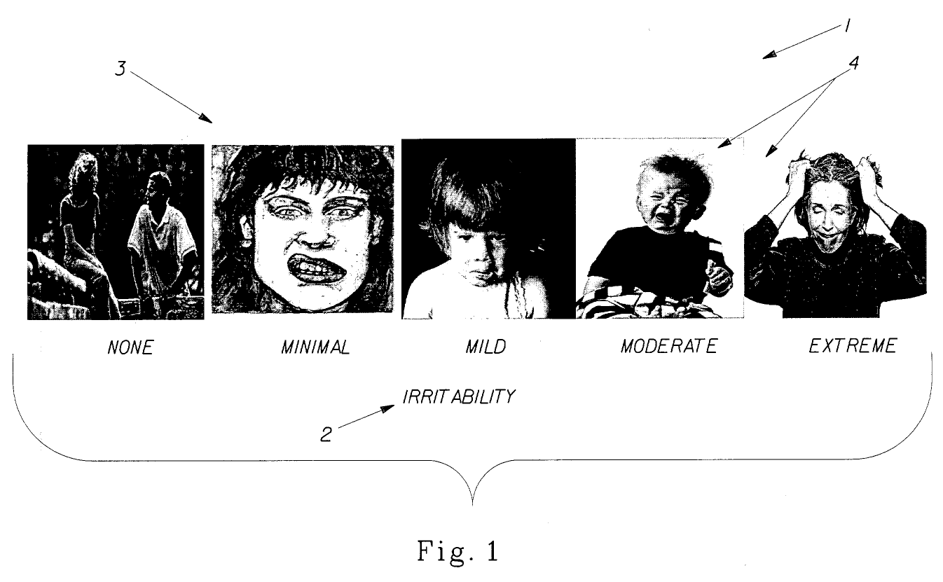

FIG. 1 shows a typical symptom rating scale 1 which is a series of individual indicia 3 for a stated or suggested symptom 2, such as the emotion shown, i.e., “irritability”. Emotional states can include symptoms such as anger, anxiety, sadness, depression, and frustration, for example. Physical states can include symptoms such as bloating, fatigue, backache, and cramps can be used for symptom 2 as well. As shown, there is a series of visual indicia 3, sometimes referred to as a picture scale, associated with the symptom rating scale 1 for any given emotion 2. The series of visual indicia 3 comprise at least two individual visual indicia 4, the individual visual indicia 4 being pictures, each picture conveying a certain level or intensity of the suggested symptom 2. Each of he individual visual indicium 4 is chosen as being suitable for conveying a certain level of intensity of the suggested emotion. It is important to note that the individual visual indicium 4 do not only convey the intensity or severity of the given emotion in a quantitative sense, i.e., “how much”, but they convey a sense of quality, i.e., “what kind”. This type of information, when recorded, for example, on a 5-point numerical scale of 0-4 greatly helps to accurately convey and understand the symptom severity levels of a person. This technique had been found to be especially helpful in facilitating efficient and effective diagnosis of symptom severity related to menstrual cycles in adult women. By recording symptom severity on a symptom rating scale 1 for a range of selected symptoms 2 daily for a sufficiently long time, e.g., one menstrual cycle, one can see cyclic differences that can result in accurate diagnosis of PMS, for example.

In one embodiment, the pictures used as individual visual indicium 4 are photographs. Photographs can very effectively communicate certain intensity levels of emotional or physical symptoms. Although FIG. 1 shows five individual visual indicia 4 in picture scale 3, there can be more or less than five individual visual indicia 4 in the picture scale 3. In general, it is believed that at least two individual visual indicia 4 should be used, preferably three and more preferably five or more pictures for each picture scale 3.

The diagnostic tool of the present invention can comprise as many different symptoms 2 as deemed necessary to understand a woman's symptom severity level. In one embodiment, the diagnostic tool included 8 emotional symptoms, including anxious, irritability, nervous, angry, sad, overwhelmed, guilt and the like. Each of the emotions had a respective picture scale 3 associated therewith. The user is directed to choose one level of intensity for each emotion based upon the choices in the picture scale. A number is associated with each visual indicium 4 so that a numerical scale can be correlated with the picture scale 3. For example, a simple numbering from 0 to 4 for a picture scale 3 having five visual indicium 4, is sufficient for analysis of symptoms throughout a menstrual cycle of an adult woman. In use, the user, after identifying the individual visual indicium 4 corresponding to her felt symptom of the suggested type, records the number (0-4) and/or word (e.g., none, minimal, mild, moderate, extreme) associated with that picture. Alternatively, the user may simply circle the appropriate individual visual indicium 4, number, and/or word). Once this is done for each of the suggested symptoms 2, the information can be used to determine the person's emotional symptom severity level. For example, the numerical level associated with each individual visual indicium 4 can be added and divided with the total number of individual visual indicia 4 used to get an average rating. This rating can be used to compare to baseline ratings, as well as for treatment of symptoms.

The emotional symptom severity level can be determined by summing up the numbers associated with the visual indicia chosen for each suggested emotion. Certain emotions may be grouped for separate evaluation. For example, negative emotions such as anger, irritability, sadness, anxiety, and depression can be grouped together to get an emotional symptom severity level for the group. However, all the numerical quantifiers corresponding to all the individual visual indicium 4 can be added to give an overall symptom rating if desired.

The symptom severity rating(s) produced by the diagnostic tool of the present invention can be used by doctors, clinicians, researchers, and other health professionals to perform their analysis to determine the emotional or physical state of their subjects. Better analysis of symptom severity levels leads to better treatment regimes, as well as to better products available for use in treatment regimes.

I claim:

1. A diagnostic tool for determining a state of a person, the tool comprising:
(a) at least one identified symptom; and
(b) a series of visual indicia corresponding to each of said at least one identified symptom.

2. The diagnostic tool of claim 1, wherein said identified symptom is a physical symptom.

3. The diagnostic tool of claim 1, wherein said identified symptom is an emotional symptom.

4. The diagnostic tool of claim 1, wherein said series of visual indicia comprise photographs.

5. The diagnostic tool of claim 4, wherein said photographs are differentiated by communicated intensity.

6. The diagnostic tool of claim 4, wherein said photographs are arranged to form a scale of increasing intensity.

7. A method of determining the emotional state of a person, said method comprising the steps of:
(a) suggesting an emotional or physical symptom;
(b) providing a series of visual indicia, each said visual indicia having a numerical value associated therewith, and each said visual indicia communicating varying intensity levels of said emotional or physical symptom; and
(c) directing the person to choose one of said indicia as representative of that person's emotional or physical symptom.

8. The method of claim 7, further comprising the step of:
(d) repeating steps (a)-(c) with a different emotional or physical symptom than the one suggested in step (a); and
(e) calculating numerical values based on said numerical values of said visual indicia.

9. The method of claim 8, further comprising the step of:
(f) determining the person's emotional or physical symptom severity level.

10. The method of claim 7, wherein said visual indicia comprise photographs.

11. The method of claim 10, wherein said photographs are arranged to form a scale of increasing intensity.
